(19) United States
(12) Patent Application Publication
(10) Pub. No.: US 2024/0027626 A1
(43) Pub. Date: Jan. 25, 2024

(54) Devices, methods, and systems for first responders

(71) Applicant: Red Line Safety, Inc., Greenwood, CA (US)

(72) Inventors: David Scott Holman, Greenwood, CA (US); David McGillicuddy, Cool, CA (US); Sashi Ono, Santa Barbara, CA (US); David Lujan, Lahaini, HI (US)

(73) Assignee: Red Line Safety, Inc., Greenwood, CA (US)

(21) Appl. No.: 18/445,148

(22) Filed: Apr. 28, 2023

Related U.S. Application Data
(60) Provisional application No. 63/473,319, filed on May 16, 2022.

(51) Int. Cl. G01S 19/17; G01N 33/00; G01J 5/08; A61B 5/0205

(52) U.S. Cl. CPC G01S 19/17; G01N 33/0031; G01J 5/0859; A61B 5/0205; G01J 2005/0077

(57) ABSTRACT

The present invention is generally directed to devices, methods, and systems for first responders. It is more specifically directed to devices, methods, and systems that collect data that can be used to improve the performance and safety of first responders. In one aspect, a device for monitoring a first responder is provided, where the device includes: a case, to which is attached an antenna, a status light, and a thermal camera; a flexible printed circuit board assembly connected to an inside portion of the case, wherein the assembly includes GPS, and at least one toxic gas sensor.

### CROSS-REFERENCE TO RELATED APPLICATION

This application claims the benefit of U.S. Provisional Patent Application No. 63/473,319, filed May 16, 2022, which is incorporated by reference herein in its entirety.

### FIELD OF THE INVENTION

The present invention is generally directed to devices, methods, and systems for first responders. It is more specifically directed to devices, methods, and systems that collect data that can be used to improve the performance and safety of first responders.

### BACKGROUND OF THE INVENTION

There have been reports of devices developed for first responders. US Pat. Pub. No. 2022/0036721, entitled “Applying Machine Intelligence for Location-Based Services to Dispatch First Responders”, for instance, allegedly discusses the following: “Techniques are described herein for applying a selective automated response to a distress call received at an emergency services system. The techniques include receiving a distress call from a user device in response to an emergency event. The distress call may be parsed to identify a location, an urgency index, and a nature of the emergency event. The distress call may be routed to an available dispatcher at a dispatcher terminal. Based at least on the location, the urgency index, and the nature of the emergency event, a response procedure to resolve the distress call may be identified. The dispatcher may communicate with a dispatched first responder at the location via a communication channel established between the dispatcher terminal and a mobile first responder terminal. The response procedure may be transmitted to the mobile first responder terminal.” Abstract.

International Pub. No. WO 2022/015724, entitled “Wearable Sensor System Configured for Alerting First Responders and Local Caregivers”, allegedly reports the following: “An apparatus comprises a processing device configured to receive health data characterizing outbreak of a disease and physiological monitoring data from wearable devices associated with users and to calculate user-specific risks of at least one of contracting and spreading the disease based on analysis of the physiological monitoring data and the health data characterizing the outbreak of the disease. The processing device is also configured to generate notifications for delivery to the users based on the user-specific risks, the notifications comprising information related to outbreak of the disease and measures for treating and mitigating spread of the disease. The processing device is further configured to modify at least a given one of the notifications to be delivered to the given user based at least in part on identifying that the given user is associated with at least one of the first responder network and the local caregiver network.” Abstract.

International Pub. No. WO 2021/096604, entitled “Device, System and Method for Role Based Data Collection and Public-Safety Incident Response” allegedly describes the following: “A device, system and method for role based data collection and public-safety incident response is provided. A computing device receives a call to report a public-safety incident. The computing device determines a role of a caller on the call, by parsing audio conversation of the caller for keywords. The role of the caller being relative to the public-safety incident such as victim, witness or first responder. The computing device retrieves, from one or more memories, data dependent on the role of the caller. The computing device controls a notification device to provide the data dependent on the role of the caller in order to provide the care-taker with relevant questions for collecting information from the caller about the incident.” Abstract.

US Pat. Pub. No. 2020/0334778, entitled “Systems and Methods for Providing Situational Awareness to First Responders”, allegedly details the following: “Systems and methods for providing situational awareness of a building to first responders are described. The system includes a server that receives and stores building information, such as a floor plan and other information. The system further includes one or more sensors that, whether or not periodically, senses situational awareness of the building and uploads that information to the server. A building identification affixed to the building allows a first responder to access the building information and situational awareness information at any time, such as during a situation or an incident, such as an emergency.” Abstract.

Despite the various reports, there is still a need in the art for new devices, methods; and systems for first responders.

### SUMMARY OF THE INVENTION

The present invention is generally directed to devices, methods, and systems for first responders. It is more specifically directed to devices, methods, and systems that collect data that can be used to improve the performance and safety of first responders.

In one aspect, a device for monitoring a first responder is provided, where the device includes: a case, to which is attached an antenna, a status light, a strobe light, an LCD screen and a thermal camera; a flexible printed circuit board assembly connected to an inside portion of the case, wherein the assembly includes GPS, a gyroscope, an accelerometer, and one, two, three, four or five toxic gas sensors.

In another aspect, a system for monitoring a first responder is provided, wherein the system comprises: a GPS main unit, a custom fitness wrist band for collection of physiological data, and a dashboard where all data collected from the GPS main unit and fitness wrist band can be displayed in real time and stored in secure servers.

In another aspect, a method of reducing injury in a first provider is provided, wherein the method comprises the steps of: collecting data using a GPS main unit and a wrist ban, wherein the GPS main unit and the wrist ban are worn by the first responder, and wherein the data includes heart rate and oxygen saturation of blood, and wherein the data is transmitted to the dashboard, where it is collected, analyzed against standards derived for fatigue and displayed, and wherein if the display indicates that the first responder is at risk of injury, he is contacted regarding next actions.

### BRIEF DESCRIPTION OF THE DRAWINGS

FIG. 1 shows one embodiment of a system according to the present invention.

FIG. 2A shows a front view of a GPS main unit of the present invention. FIG. 2B shows a view where the GPS main unit shown in FIG. 2A has been slightly turned.

FIG. 3A shows a back view of a GPS main unit of the present invention. FIG. 3B shows a view where the GPS main unit shown in FIG. 3A has been slightly turned.

FIG. 4A shows a top view of a GPS main unit of the present invention. FIG. 4B shows a bottom view of a GPS main unit of the present invention.

FIG. 5 shows an outside view of a flex board assembly of the GPS main unit of the present invention.

FIG. 6 shows an inside view of the flex board assembly of the GPS main unit of the present invention.

FIG. 7 shows an example of a wrist band used in a system of the present invention.

FIG. 8 shows an example of a dashboard used in a system of the present invention.

### DETAILED DESCRIPTION OF THE INVENTION

When responding to an emergency, a first responder is oftentimes exposed to a hazardous environment. The nature of the hazardous environment varies, for example, from exposure to toxic gases, to exposure of non-gaseous toxins, to exposure of dangerous site aspects (e.g., unstable structures, electric shocks and structures that present falling risks). The hazardous environments also result in physical and psychological responses that can be detrimental to the first responder. Stress, emotional fatigue, and physical fatigue are examples of responses that can result in a first responder's harm both on the job in the environmental hazard and after the job is done.

A concept behind the present invention is that the collection and analysis of data from a hazardous, or potentially hazardous, environment can help to minimize exposure of a first responder to hazards. The minimization of exposure would further limit detrimental emotional and physical responses by the first responder.

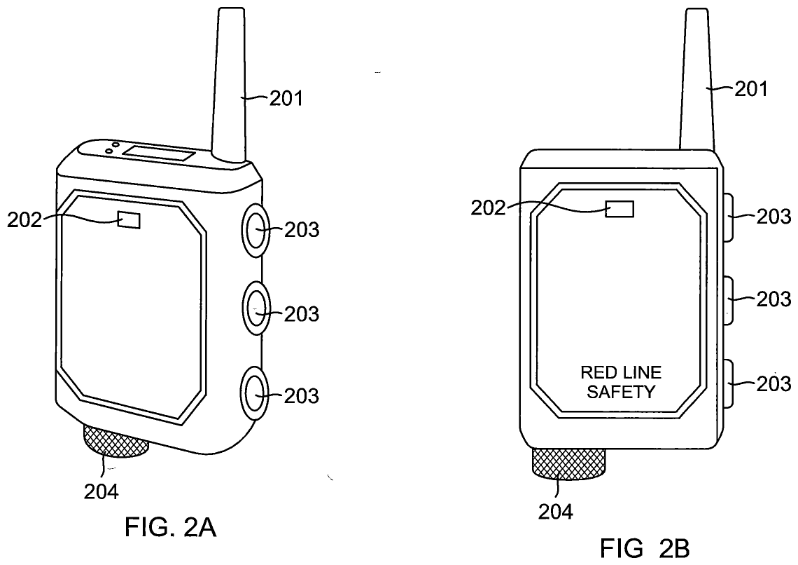

In one aspect, the present invention provides a GPS main unit. The unit is portable, and typically cloud-based. It provides for the collection and distribution of personal, atmospheric and environmental data. One example of the GPS main unit is shown in FIGS. 2A, 2B, 3A, 3B, 4A, 4B, 5 and 6. FIG. 2A shows a front view of the GPS main unit. Antenna 201 (e.g., 900 mhz LoRaWAN) is connected to a case 200. Case 200 further includes a grid eye thermal cameral 202 (e.g., 8×8 grid forward facing heat signature), three user buttons 203 (e.g., on-off, mode select—up, down, enter, and SOS), and a removable, rechargeable lithium-ion battery cap 204 (e.g., 18650 lithium-ion battery cap). FIG. 2B shows a view where the GPS main unit shown in FIG. 2A has been slightly turned, and elements 201-204 are visible.

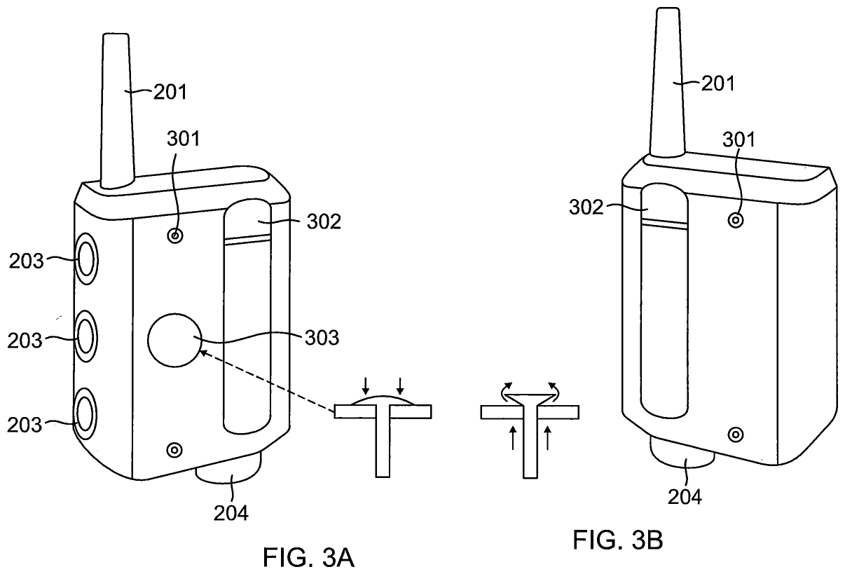

FIG. 3A shows a back view of a GPS main unit of the present invention. Antenna 201 is connected to case 200. Three user buttons 203 are mounted on the side of case 200 and removable, rechargeable lithium-ion battery cap 204 is attached to the bottom of case 200. Case assembly screw 301 holds case 200 and its various components in place. Belt clip 302 is shown mounted to the back of case 200. Umbrella valve 303, which releases pressure that builds up within case (add number), is at the center left of case (add number). FIG. 3B shows a view where the GPS main unit shown in FIG. 3A has been slightly turned, and elements 201, 204, 301 and 302 are visible.

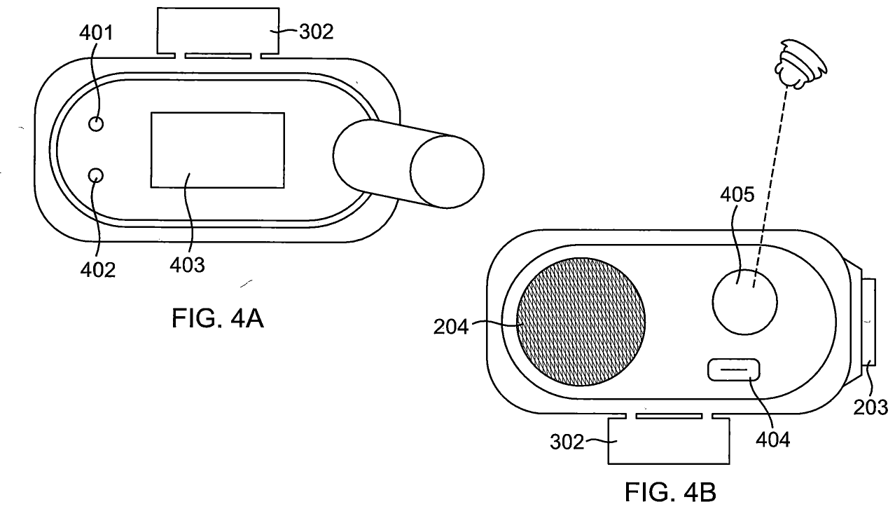

FIG. 4A shows a top view of a GPS main unit of the present invention. A top view of belt clip 302 is shown. Status light 401 (on, cell connect, LoRaWAN connect), strobe light 402, which flashes for visual notification when an SOS feature is activated, and screen 403 (e.g., color LCD screen, sensor readings, battery status) are shown on the top of case 200. FIG. 4B shows a bottom view of a GPS main unit of the present invention, where user button 203, battery cap 204, and belt clip 302 are visible. USB “C” charging port 404 and a Donaldson's Snap-Fit enclosure protection vent 405, which provides protection from dust, dirt, water, oils, and liquids while allowing pressure equalization, are shown at the bottom of case 200.

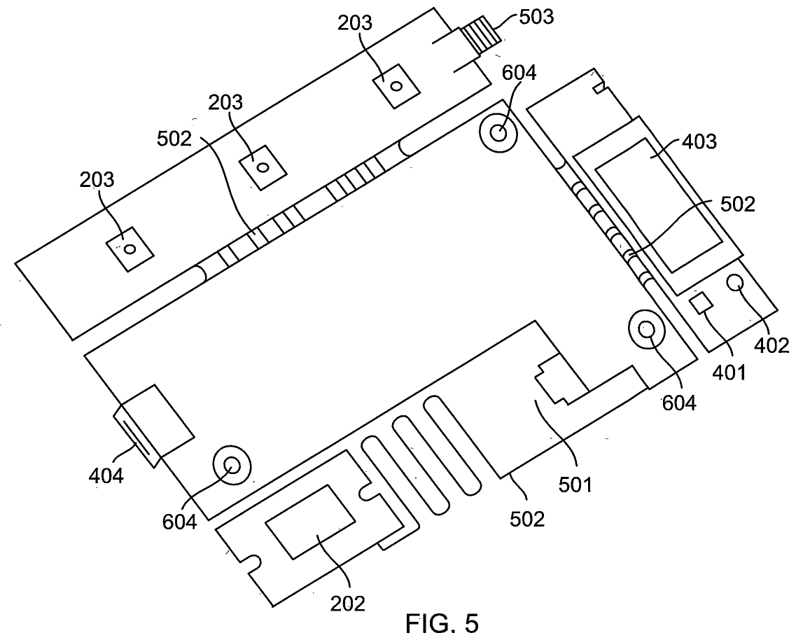

FIG. 5 shows an outside view of a flexible printed circuit board assembly of the GPS main unit of the present invention. 202 is a grid eye thermal camera embedded in the flexible printed circuit board. The point of contact for the three user buttons 203 on the circuit board is shown. Status light 401, strobe light 402 and screen 403 are embedded in the circuit board and charging port 404 is attached to the edge of a board distal to screen 403. Lithium-ion battery port 501 is attached to an area of the circuit board distal to user buttons 203. Flex cables 502 attach printed circuit boards together to form the assembly, and antenna 503 is adjacent to the three user buttons 203. PCB mounts 604 hold the circuit board to the interior of case 200.

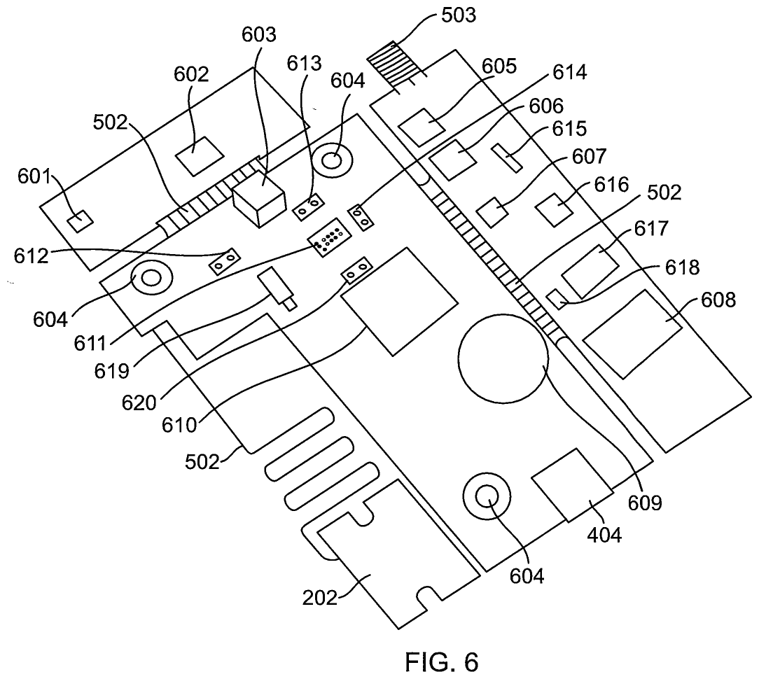

FIG. 6 shows an inside view of the flexible printed circuit board assembly of the GPS main unit of the present invention. USB “C” charging port 404 is connected to the flexible printed circuit board. Flex cables 502 attach printed circuit boards together to form the assembly, and antenna 503 is attached to the board at a position distal to charging port 404. Piezo component 601 is attached to the circuit board proximal to component 602, which provides multiprotocol support for Bluetooth connectivity (e.g., NRF 52840 with full protocol concurrency). Carbon dioxide sensor 603 (e.g., SCD41-D-R1 40PPM 12C SMD) is attached to the printed circuit board between PCB mount holes 604. Microcontroller 605 (e.g., STM32WL55CCU6—LPWAN Dual Core ARM Cortex-M4/M0) is connected to the printed circuit board between antenna 503 and microcontroller 606 (e.g., ESP 32—Bluetooth, WiFi 802.11b/g/n). GPS module 607 (e.g., UBLOX GPS—ZOE-M8B SiP ultra small, super low power u-blox M8 GNSS SiP) is attached between microcontroller 606 and embedded modem and GPS receiver 608 (e.g., NRF 9160 LTE-M/NB-IoT/GPS Module 700 MHz to 2200 MHz, 3.3V to 5.5V LGA). Oxygen sensor 609 (e.g., O2 Sensor Series 4 Oxygen Sensor 0-25%) is embedded adjacent to CO sensor 610 (e.g., Electrochemical carbon monoxide sensor TGS5141 CO Figaro [CO 0-10000 ppm]). Multi gas sensor 611 (e.g., Multi Gas Sensor MICS-6814—SMD Carbon Monoxide/Nitrogen Dioxide/Ammonia triple sensor) is embedded between carbon dioxide sensor 603 and CO sensor 610. Hydrogen sulfide sensor 612, methane sensor 613, and hydrogen cyanide sensor 614 are also shown on the printed circuit board. WiFi/Bluetooth 615, GPS 616, and cellular card 617 are connected to the printed circuit board between antenna 503 and GPS receiver 608. BME 680 (618), which senses air temperature, barometric pressure, humidity, and total volatile organic compounds, is shown on the printed circuit board between cellular card 617 and GPS receiver 608. A vibration motor 619 is shown adjacent to multi gas sensor 611, and radioactivity sensor 620 is adjacent to the CO sensor.

Nonlimiting examples of sensors that can be installed on the printed circuit board (including replacement of sensors listed above) include: carbon dioxide sensor; carbon monoxide sensor; hydrogen sulfide sensor; hydrogen cyanide sensor; hydrogen chloride sensor; polycystic aromatic hydrocarbons sensor; liquid propane gas sensor; total volatile organic compounds sensor; lower explosive limits sensor; and, upper explosive limits sensor. Nonlimiting examples of atmospheric sensors that can be installed on the printed circuit board (including replacement of sensors listed above) include: air temperature; oxygen concentration; humidity; barometric pressure; xenon; nitrogen; hydrogen; helium; krypton and carbon dioxide. Nonlimiting examples of toxic gas sensors that can be installed on the printed circuit board (including replacement of sensors listed above) include: carbon dioxide; carbon monoxide; hydrogen cyanide; ammonia; hydrogen chloride; sulfur dioxide; hydrogen sulfide; oxides of nitrogen; PAH's; benzene; toluene; styrene; metals; dioxins; LPG; butane; methane; alcohol; natural gas; TVOC; LEL; and, UEL.

Various features of the GPS main unit include: GPS for asset location tracking on a variety of maps (USGS topo, street maps, Google maps); SOS (manual and automatic) for lost/trapped/injured or first responder in distress; gyroscope, which identifies when a first responder has experienced a high velocity rotation; accelerometer, which identifies when a first responder experiences a high velocity movement; cellular/LTE for primary connectivity; WiFi for additional connectivity problems; Bluetooth for additional connection options; mesh networking, which allows the need for only one first responder's system to have connectivity; toxic gas sensors, used for the detection of a hazardous environment; atmospheric sensors, which detect local air quality; LoRaWan, or long range communication platform; radiological sensor, which detects if the area is radiological positive; vibra alert, which vibrates for physical notification when from data/SOS/evacuation order indicates; strobe light, which flashes for visual notification when the SOS feature is activated; forward facing thermal camera, for transmission of infrared video of heat signatures; tag in/tag out programing, where first responders are auto-marked inside or outside a structure based on their location; external charging port; removable rechargeable battery; water and hear resistant enclosure.

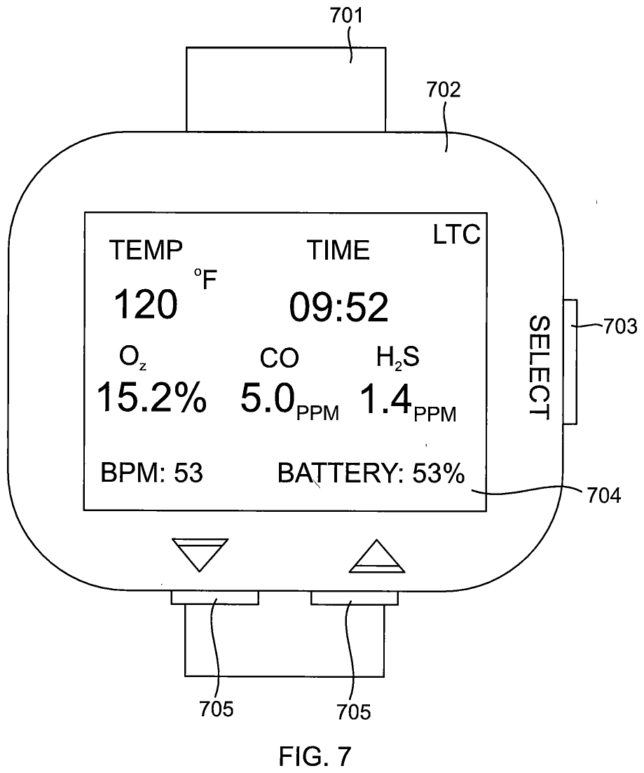

FIG. 7 shows an example of a wrist band used in a system of the present invention. Band strap 701 is connected to exterior case 702. A mode selection button 703 is connected to the side of case 702. An LCD display 704 is attached to the surface of exterior case 702, and mode display buttons 705 are adjacent to display 704.

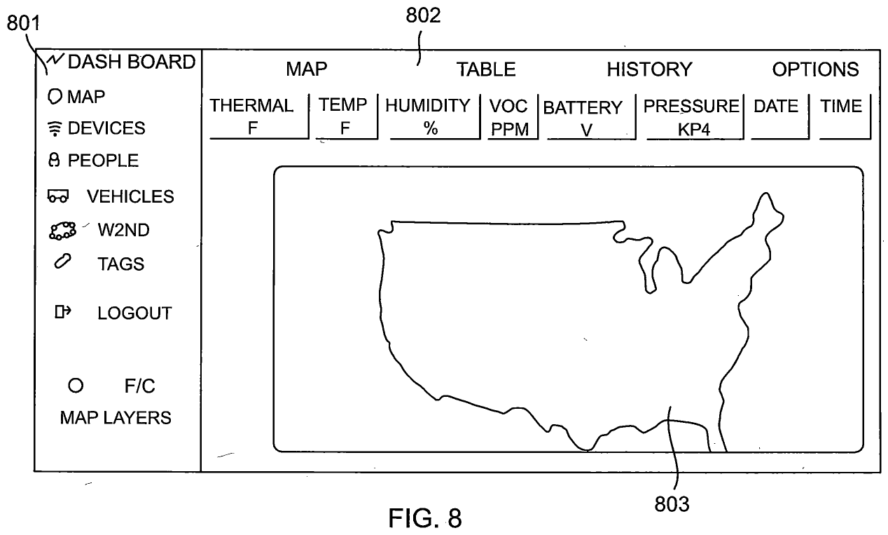

FIG. 8 shows an example of a dashboard used in a system of the present invention. Selectable filters 801, sensor data 802 and a map display 803 are shown on the surface of the dashboard.

In another aspect, the present invention provides a data collection and tracking system for first responders. The system typically includes three components: 1) the GPS main unit discussed above; 2) a custom fitness wrist band for the collection of physiological data; 3) a dashboard where all data collected from the GPS main unit and fitness wrist band can be displayed in real time and stored in secure servers.

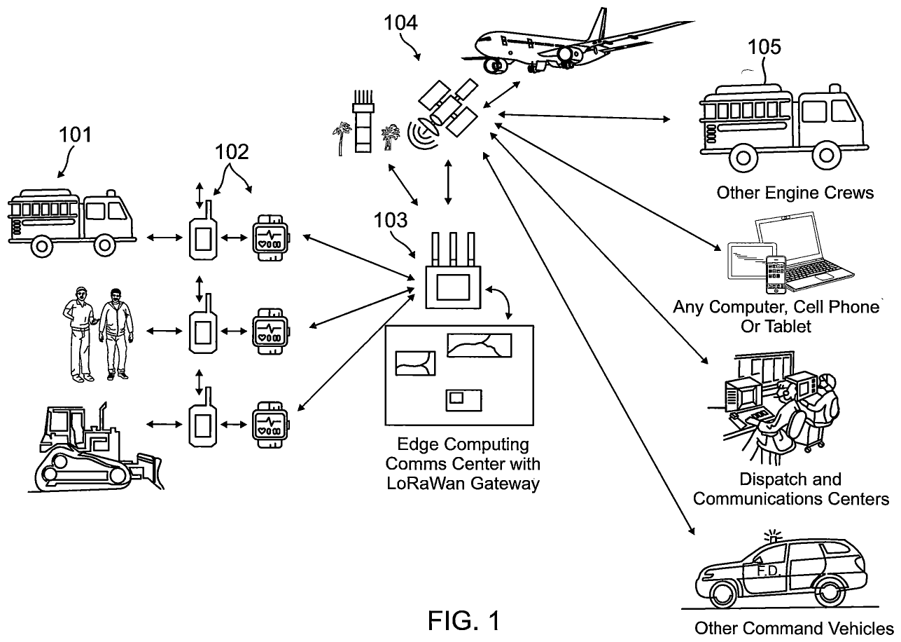

FIG. 1 shows one embodiment of a system according to the present invention. Resource 101 equipped with IMSAFE (personnel, vehicles, aircraft, heavy equipment operators, etc.) is shown. Two wearables of IMSAFE 102 are shown connected to resource 101, the Hub and the Band that gathers sensor data. The data collection system 101 and 102 transmits collected data and sends it to an LoRaWAN gateway 103. At gateway 103, edge computing takes place to refine and repackage the data. Gateway 103 then allows the data to be viewed in real-time at the location and simultaneously sends the data to cloud 104 via cellular or satellite connectivity 104. Once in the cloud 104, the data collected from resources 102 can be shared with other resources that are connected to the cloud 104 in real-time. The data is also simultaneously stored in secured servers for further use by the individual resource 101, fire department administrators, medical researchers or Red Line Safety, Inc. End users 105 use the real-time data collected from resources 101. This includes other crews, other command vehicles, anyone with a device connected to the cloud 104 and communication centers.

The custom fitness wrist band monitors a first responder's physiological status. It provides a full spectrum of health and wellness data with applied algorithms for the first responder around the clock. Aspects of the wrist band include: an LCD display of critical data; HR display of heart rate in beats per minute; PPG display showing the oxygen saturation of blood; Skin Temperature, arrived at through machine learning to show whether a body is overheated or febrile; vibra alert to alert the first responder when alarms are activated.

The dashboard provides for the display of data gathered from the GPS main unit and the fitness wrist band. Data displayed by the dashboard includes, without limitation, the following: first responder location; first responder physiological status; atmospheric conditions; location and proximity to target hazards; hazardous environments; vehicle location and status; customizable map overlays; search options; large variety of filters; specific search bar; drawing tools; on screen timers/incident clock/PAR clock.

In one aspect, a device for monitoring a first responder is provided, where the device includes: a case, to which is attached an antenna, a status light, a strobe light, an LCD screen and a thermal camera; a flexible printed circuit board assembly connected to an inside portion of the case, wherein the assembly includes GPS, a gyroscope, an accelerometer, and one, two, three, four or five toxic gas sensors.

In another aspect, a system for monitoring a first responder is provided. The system includes a GPS main unit, a custom fitness wrist band for collection of physiological data, and a dashboard where all data collected from the GPS main unit and fitness wrist band can be displayed in real time and stored in secure servers.

In another aspect, a method of obtaining, analyzing and displaying data related to a first responder is provided. Data is collected by the GPS main unit and the wrist band, both of which are worn by a first responder. The data is transmitted to the dashboard, where it is collected, analyzed and displayed.

In another aspect, a method of reducing fatigue in a first responder is provided. Data is collected by the GPS main unit and the wrist band—both of which are worn by a first responder—where the data includes infrared video of heat signatures, heart rate and oxygen saturation of blood. The data is transmitted to the dashboard, where it is collected, analyzed against standards derived for fatigue and displayed. If the display indicates that the first responder is experiencing fatigue, he is contacted regarding next actions.

The biometric data collected by the GPS main unit and wrist band can be used to help ensure first responders are properly monitored during emergency operations. This data will allow for the ability to ensure adequate rehabilitation of the first responder before leaving the emergency scene. By improving their recover, first responders are more capable of going to another emergency with less fatigue and less likely to be injured.

One way to determine whether fatigue experienced by first responders has been reduced is to compare fatigue rates between groups of first responders who generally are exposed to a similar, or the same, risk of fatigue over a similar, or the same, period of time. See Yung et al. “Fatigue Risk Management for First Responders: Current Landscape of Perspectives, Policies, and Practices” https://www-assets.conestogac.on.ac. One group receives the benefit of a system according to the present invention; the other is not. The group receiving the benefit of the system typically will have a reduction in fatigue of at least 5 percent (e.g., reduction of five percent to ten percent). In other cases, the reduction in fatigue will be at least 10 percent (e.g., 10 percent to 20 percent). In still other cases, the reduction in fatigue will be at least 20 percent (e.g., 20 percent to 30 percent), at least 30 percent (e.g., 30 percent to 40 percent), or at least 40 percent (e.g., 40 percent to 50 percent).

In another aspect, a method of reducing injury experienced by a first responder is provided. Data is collected by the GPS main unit and the wrist band—both of which are worn by a first responder—where the data includes infrared video of heat signatures, toxic gas concentrations in the environment and oxygen saturation of blood. The data is transmitted to the dashboard, where it is collected, analyzed against standards derived for risk of injury and displayed. If the display indicates that the first responder is at risk for injury, he is contacted regarding next actions.

If it is determined that the first responder is at risk of medical emergency, or indeed injured, not only will their exact location be known; the location and qualifications of assistance resources will be known, since all operators will be displayed in real time on the dashboard. During an SOS, or emergency operation, the tabs and filter features of the dashboard may be used in a manner that will provide situational focus for the involved resources and incident priorities.

One way to determine whether injuries experienced by first responders has been reduced is to compare injury rates between groups of first responder's who generally are exposed to a similar, or the same, risk of injury over a similar, or the same, period of time. See, Dannenberg and Fowler, Injury Prevention 1998; 4:141-147. One group receives the benefit of a system according to the present invention; the other is not. The group receiving the benefit of the system typically will have a reduction in injury of at least 5 percent (e.g., reduction of five percent to ten percent). In other cases, the reduction in injury will be at least 10 percent (e.g., 10 percent to 20 percent). In still other cases, the reduction in injury will be at least 20 percent (e.g., 20 percent to 30 percent), at least 30 percent (e.g., 30 percent to 40 percent), or at least 40 percent (e.g., 40 percent to 50 percent).

In another aspect, a method of reducing the rate of cancer diagnoses in first responders is provided. Data is collected by the GPS main unit and the wrist band—both of which are worn by a first responder—where the data includes toxic gas concentrations in the environment and radioactivity levels. The data is transmitted to the dashboard, wherein it is collected, analyzed against standards derived for cancer risk and displayed. If the display indicates that the first responder is at risk for cancer—e.g., significant smoke inhalation—he is contacted regarding next actions.

It is important to note that even though first responders (e.g., firefighters) wear industry approved personal protective equipment (PPE), the PPE is considered only a partial barrier to the many dangers of atmospheric and toxic exposure encountered by first responders. It is not 100% protection. Having a digitalized record of exposure allows a better understanding of what might need to be performed to better prepare and protect the first responders—e.g., enhanced post exposure on-scene first responder detoxification, proper post exposure PPE cleaning, post exposure medical follow-up, etc.

One way to determine whether cancer diagnoses experienced by a first responder has been reduced is to compare cancer diagnoses between groups of first responders who generally are exposed to a similar, or the same, cancer risk over a similar, or the same, period of time. See, “Reducing Firefighter Cancer Risks”, https://www.powerdms.com/policy-learning-center/-reducing-firefighter-cancer-risks. One group receives the benefit of a system according to the present invention; the other is not. The group receiving the benefit of the system typically will have a reduction in cancer diagnoses of at least 5 percent (e.g., reduction of five percent to ten percent). In other cases, the reduction in cancer diagnoses will be at least 10 percent (e.g., 10 percent to 20 percent). In still other cases, the reduction in cancer diagnoses will be at least 20 percent (e.g., 20 percent to 30 percent), at least 30 percent (e.g., 30 percent to 40 percent), or at least 40 percent (e.g., 40 percent to 50 percent).

In another aspect, a method of reducing heart disease in first responders is provided. Data is collected by the GPS main unit and the wrist band—both of which are worn by a first responder—where the data includes toxic gas concentration in the environment, heart rate and oxygen saturation of blood. The data is transmitted to the dashboard, where it is collected, analyzed against standards derived for risk of heart disease and displayed. If the display indicates that the first responder is at risk for heart disease, he is contacted regarding next actions.

First responders (e.g., firefighters) often go from one emergency to the next. Having this data in real time allows on scene managers to know what steps must be taken to properly prepare the first responder to respond to the next emergency. This is where the value of proper rehabilitation applies. In addition, comparing this real time data to an individual's historical data, which would be accessible while at the scene of an emergency, will allow for a better understanding of when each individual is properly conditioned to return to work safely.

One way to determine whether heart disease experienced by a first responder has been reduced is to compare heart disease between groups of first responders who generally, are exposed to a similar, or the same, risk of heart disease over a similar, or the same, period of time. See Kales et al. “Emergency Duties and Deaths from Heart Disease Among Firefighters in the United States” New England Journal of Medicine 2007 March; 256(12) 1207-1215. One group receives the benefit of a system according to the present invention; the other is not. The group receiving the benefit of the system typically will have a reduction in heart disease of at least 5 percent (e.g., reduction of five percent to ten percent). In other cases, the reduction in heart disease will be at least 10 percent (e.g., 10 percent to 20 percent). In still other cases, the reduction in heart disease will be at least 20 percent (e.g., 20 percent to 30 percent), at least 30 percent (e.g., 30 percent to 40 percent), or at least 40 percent (e.g., 40 percent to 50 percent).

In another aspect, a method of reducing chronic respiratory diseases in first responders is provided. Data is collected by the GPS main unit and the wrist band—both of which are worn by a first responder—where the data includes toxic gas concentration in the environment and oxygen saturation of blood. The data is transmitted to the dashboard, where it is collected, analyzed against standards derived for risk of chronic respiratory disease and displayed. If the display indicates that the first responder is at risk for chronic respiratory disease, he is contacted regarding next actions.

The main unit and the wrist band work together to provide real-time data for the environment a first responder (e.g., firefighter) is working in and how his body is performing and reacting in a given work environment. By knowing these important data points on the outside of a first responder's PPE, and knowing the data points of how the first responder is physiologically responding on the inside of the PPE, one can differentiate whether a first responder is in crisis or simply working hard. This real-time data can be compared to historical data to help determine the level of stress a first responder is experiencing. Having such data stored will help guide the first responder to improved health and wellness over the course of his career.

On a larger scale, having this type of data, cleaned of all personal information, could help researchers better understand the effects of being a first responder and help develop focused studies to prevent, reduce and treat first responder (e.g., firefighter) illnesses.

One way to determine whether chronic respiratory disease experienced by a first responder has been reduced is to compare chronic respiratory disease incidence between groups of first responders who generally are exposed to a similar, or the same, risk of chronic respiratory disease over a similar, or the same, period of time. See Gupta et al. “Smoke Inhalation Injury: Etiopathogenesis, Diagnosis, and Management” Indian J Crit Care Med 2018 March; 22(3):180-188.

One group receives the benefit of a system according to the present invention; the other is not. The group receiving the benefit of the system typically will have a reduction in chronic respiratory disease of at least 5 percent (e.g., reduction of five percent to ten percent). In other cases, the reduction in chronic respiratory disease will be at least 10 percent (e.g., 10 percent to 20 percent). In still other cases, the reduction in chronic respiratory disease will be at least 20 percent (e.g., 20 percent to 30 percent), at least 30 percent (e.g., 30 percent to 40 percent), or at least 40 percent (e.g., 40 percent to 50 percent).

### Examples

A test subject firefighter was fitted with a Maxim Wrist band and carried a cell phone in his pocket to record physiological data during his time in the burn area. The VMB was designed to burn away vegetation in a controlled manner. Units mostly in holding status but occasionally engaged in firefighting operations to control designed burn areas. Device removed from firefighter at the end of the day. Researchers immediately noticed a maximum heart rate of 208 bpm approximately 30-45 minutes prior to dropping off the equipment. An equipment diagnostic was performed. The technician said the band seemed to be in proper working order and had produced consistent data throughout the 3-hour procedure. The firefighter was interviewed later in the day to determine what the circumstances were that may have contributed to the extremely high heart rate. The firefighter said that at approximately 11:45, he encountered heavy smoke with high heat conditions and was having trouble breathing and coughing. He also stated that he had a lot of “snot running down my nose and all over my face at the time”. This timeline is consistent with the data collected per the researcher assigned to the subject firefighter. Later in the interview, the firefighter revealed that he had a tough time passing his pre-employment physical treadmill test because every time he was on the treadmill, he would have runs of asymptomatic SVT. After 3 failures, the subject firefighter had a doctor perform an ablation to his heart that seemed to resolve his cardiac abnormality. The firefighter was later able to pass his pre-employment physical and was hired. The subject was advised of the scientific findings, and with the information has the option to follow up with his doctor. The test subject received treatment from his personal physician and is SVT free. He will have regular, follow-up visits with his physician. This single case demonstrates the value of monitoring the physiological data of first responders (e.g., firefighters) during their work cycles. The data received and given to the test subject allowed him to seek medical attention, significantly lowering his risk of serious injury, or even death, under similar conditions in the future.

1. A device for monitoring a first responder, wherein the device comprises: a case, to which is attached an antenna, a status light, and a thermal camera; a flexible printed circuit board assembly connected to an inside portion of the case, wherein the assembly includes GPS, and at least one toxic gas sensor.

2. The device according to claim 1, wherein the device further comprises a gyroscope and an accelerometer.

3. The device according to claim 1, wherein the device further comprises a strobe light and an LCD screen.

4. The device according to claim 1, wherein the device further comprises two, three or four toxic gas sensors.

5. The device according to claim 4, wherein the device further comprises a gyroscope, an accelerometer, a strobe light and an LCD screen.

6. A system for monitoring a first responder, wherein the system comprises: a GPS main unit, a custom fitness wrist band for collection of physiological data, and a dashboard where all data collected from the GPS main unit and fitness wrist band can be displayed in real time and stored in secure servers.

7. The system according to claim 6, wherein the GPS main unit comprises: a case, to which is attached an antenna, a status light, and a thermal camera; a flexible printed circuit board assembly connected to an inside portion of the case, wherein the assembly includes GPS, and at least one toxic gas sensor.

8. The system according to claim 6, wherein the system analyzes the data collected from the GPS main unit and fitness wrist band to determine whether the first responder is in a hazardous environment.

9. The system according to claim 7, wherein the GPS main unit further comprises: a gyroscope, an accelerometer, a strobe light, and an LCD screen.

10. The system according to claim 8, wherein the system further communicates with the first responder, or another person who is monitoring the first responder, regarding the presence of a hazardous environment.

11. A method of reducing injury in a first responder, wherein the method comprises the steps of: collecting data using a GPS main unit and a wrist ban, wherein the GPS main unit and the wrist ban are worn by the first responder, and wherein the data includes heart rate and oxygen saturation of blood, and wherein the data is transmitted to the dashboard, where it is collected, analyzed against standards derived for fatigue and displayed, and wherein if the display indicates that the first responder is at risk of injury, he is contacted regarding next actions.

12. The method according to claim 11, wherein the data further includes thermal camera images.

13. The method according to claim 11, wherein the data further includes level of exposure to toxic gases.

14. The method according to claim 11, wherein the next actions are rehabilitation before leaving an emergency scene.

15. The method according to claim 12, wherein the data further includes level of exposure to toxic gases, and wherein the next actions are rehabilitation before leaving an emergency scene.
